(19) United States
(12) Patent Application Publication
(10) Pub. No.: US 2005/0158709 A1
(43) Pub. Date: Jul. 21, 2005

(54) Methods for determinging the influence of protein binding on antiretroviral activity

(76) Inventors: Hilde Azijn, Leuven (BE); Piet Tom Bert Paul Wigerinck, Terhagen (BE); Marie-Pierre T M M G De Bethune, Everberg (BE)

Correspondence Address: Dianne B Elderkin;Woodcock Washburn, One Liberty Place-46th Floor, Philadelphia, PA, 19103, US

(21) Appl. No.: 10/504,675

(22) PCT Filed: Feb. 21, 2003
(86) PCT No.: PCT/EP03/01846

Related U.S. Application Data
(60) Provisional application No. 60/358,307, filed on Feb. 22, 2002.

(51) Int. Cl. C12Q 1/70

(52) U.S. Cl. 435/5

(57) ABSTRACT

The present invention relates to methods for determining the influence of human plasma or serum protein binding on antiretroviral activity at physiologically achieved conditions, by using specific virus strains in a cell-based antiviral assay.

This application claims priority benefit of U.S. Provisional Application No. 60/358307 Feb. 22, 2002, the contents of which are expressly incorporated by reference herein.

### BACKGROUND OF THE INVENTION

Following administration, drugs are transported in biological fluids (e.g. in blood) partly in solution as free drug and partly bound to blood components (e.g., plasma or serum proteins, blood cells). The physiologically active substances are in equilibrium between a free form and a form bound to endogenous ligands present in the same fluids (see reviews by Kremer, et al. Pharmacol Rev. 1988, 40:147). Only free drug is available for passive diffusion to target tissue sites where the desired biological activity may take place. When compared to the total-substance level, the free drug concentration is more closely related to drug concentration at the active site, to drug effects, and to clinical effectiveness. As such it is generally considered that the unbound fraction of drug is pharmacologically active (Levy, G.: Effect of plasma or serum protein binding of drugs on duration and intensity of pharmacological activity. J. Pharm. Sci. 65, 1264-1265 (1976)) and that the bound fraction is not immediately available for distribution through the body or for certain routes of elimination.

Theoretically, there is a direct proportional relationship between the total plasma concentration and the unbound fraction as long as the saturation level of the finite number of binding sites on the plasma proteins is not reached. For most drugs given within the normal clinical therapeutical ranges, the protein concentration to which a drug may bind is much higher and therefore, saturation of potential binding sites is hardly ever exceeded or even approached.

However, in practice, there are many exceptions to this rule, such as, for instance, salicylate, disopyramide, phenylbutazone, naproxen, valproic acid, whose total plasma concentrations exhibit an inversely linear relationship with the binding to plasma or serum proteins, i.e. the higher the total plasma concentration, the lower the bound fraction.

Thus, binding to plasma or serum proteins may have significant changes in the unbound fraction of certain drugs, which may further translate in effects on the distribution, pharmacological activity and rate of elimination of the same.

Along this line, large changes in kinetic parameters associated to changes in plasma or serum protein binding are frequently cited to predict some important alteration in clinical effect. See Pharmacokinetic and Pharmacodynamic Data Analysis, Concepts & Applications, J. Gabrielsson and D. Weiner, 3^{rd} Ed, Swedish Pharmaceutical Press. However, when changes in binding are associated with clinical effects, it has almost always been found that this is the result of a change in unbound drug clearance caused by a mechanism quite independent of plasma or serum protein binding (Holford NHG. Clin. Pharmacokinetic. 29: ppl 139 (1995)). Winter et al. (Basic Clinical Pharmacokinetics, 3^{rd} ed., Applied Therapeutics, (1994)) concluded that there is little evidence demonstrating that monitoring unbound drug levels improves the correlation between the plasma or serum concentration and the pharmacologic effect or therapeutic outcome. See also Levy G. (In Drug-Protein binding. Edited by Reidenberg MM, Errill S. Praeger, New York, 1986).

Nevertheless, the question whether plasma or serum protein binding had an effect on in vivo activity of anti-HIV compounds has recently heightened a great interest in the investigations of effects of serum proteins on activity and pharmacokinetics of antiviral compounds in vitro (Billello et al. 1995, J. Infect. Dis. 171:546-551; Bilello et al. 1996, Antimicrobiol. Agents Chemother. 40:1491-1497; Lazdins et al. 1996, J. Infect. Dis. 175:1063-1070; Kiriyama et al. 1996, Biopharmac. Drug Dispos. 17:739-751; Zhang et al. 1999, J. Infect. Dis. 180:1833-1837; Jones et al. 2001, Br J Clin Pharmacol. 51:99-102; Kageyama et al. 1994, Antimicrob Agents Chemother. 22:499-506), and in vivo (Sadler et al. 2001, Antimicrob. Agents Chemother. 45:852-856).

In general, it has been asserted that high levels of protein binding of antivirals lead to poor clinical efficacy. As illustration, the study in vitro by Bilello et al. (1995, J. Infect. Dis. 171:546-551; 1996, Antimicrobiol. Agents Chemother. 40:1491-1497) demonstrated that the antiviral efficacy of two HIV protease inhibitors (PIs), A77003 and A80978, decreased as the concentration of α₁-acid glycoprotein (AAG) was increased and that the inhibition of HIV protease was highly correlated with the amount of intracellular inhibitor. Similarly, the clinical significance of these effects in vitro was shown by the lack of clinical efficacy of the HIV PI SC-52151, which has potent antiretroviral activity in vitro but insufficient activity in vivo, because extensive protein binding prevented intracellular diffusion (Fischl et al. J Acquir Immune Defic Syndr Hum Retrovirol 1997, 15:28-34).

Despite of a considerable literature regarding the effect of protein-binding on antiretrovirals, most methods for calculating the effect of plasma or serum protein binding, are focused on quantifying the unbound fraction of drug by employing common physico-chemical protocols, including equilibrium dialysis, ultrafiltration, ultracentrifugation, and gel filtration; and indirect methods such as determination of drug in saliva, or measurement of blood/plasma or serum or erytlhrocyte/plasma or serum drug concentration ratios. Thus, existing methods do not allow to measure the plasma protein binding of drugs with physiological conditions.

Although it has been argued that the free drug concentration, or unbound drug levels, is more closely related to drug concentration at the active site, to drug effects, and to clinical effectiveness, the monitoring of unbound drug levels still fails to find a relationship between plasma or serum concentration and the pharmacologic effect or therapeutic outcome, see Winter and Levy et al. above.

In view of the clinical significance and the medical need to pharmacokinetically characterize HIV inhibitors, a convenient and reliable method to measure the functional effect of protein binding on the efficacy of drugs is the subject of this invention. Accordingly, the present invention provides a method for measuring the influence of plasma or serum protein binding on antiretroviral therapy by use of a cell-based antiviral assay. This method has proved successful in finding a relationship between drug plasma or serum concentrations in the presence of plasma or serum proteins, with pharmacologic effects. This correlation is possible when using specific viral strains as means for testing at drug concentrations that fall within the range of drug plasma concentrations present at physiological conditions, i.e. in patients. By using such viral strains, one is able to test at a physiological and at a measurable range of effective concentrations, that is, in the physiological zone where a dose-response curve may be obtained.

The method of the present invention is additionally advantageous as it is a reliable simplification of the in vivo environment of an antiviral agent, as well as an approximate reproduction of the complexity of human plasma or serum It further takes into consideration the in vivo equilibrium and/or ready-state kinetics experienced by the antiviral drugs with the viruses. It additionally ponders the different mechanisms and stages of binding kinetics exhibited by the drugs with their ligands, i.e. the accumulation kinetics of concentration-dependent binding to tissues, the linear (constant free fraction) or concentration-dependent (increasing free fraction with increasing drug concentration) binding to plasma or serum proteins.

The present invention provides a method for pharmacokinetically characterizig HIV inhibitors in the presence of plasma or serum proteins, that is by determining therapeutic amounts and subsequent dosage regimens, resulting in more accurate and effective therapeutic amounts and dosage regimens, ultimately translating in an improved treatment for HIV infected patients.

The method subject of this invention allows as well for an improved preclinical evaluation and selection of new antivirals for future clinical development.

Furthermore, often there may be competition between drugs in plasma or serum protein binding, in which agents that are bound tightly, such as coumarin anticoagulants, macrolide or lincosamide antibiotics that bind tightly to α₁-acid glycoprotein (AAG), are able to displace less tightly bound compounds from their binding sites and thus can increase the free form of the drug and improve the biological efficacy (Sommadossi, et al., 1998 U.S. Pat. No. 5,750,493). Therefore, the present invention provides as well a method for selecting compounds that competitively bind with plasma or serum proteins, said selection being useful for co-administering agents to compete for plasma or serum protein binding, so that an increase of the free plasma or serum concentration of antiretrovirals is achieved. Alternatively, highly potent HIV inhibitors, eventually with a narrow therapeutic range, may further benefit from the co-administration of compounds that enhance binding of HIV inhibitors with plasma or serum proteins, so that their working concentrations are maintained free from toxic levels, thus preventing overmedication. Consequently, the present invention includes as well a method for selecting compounds that enhance binding of antiretrovirals with plasma or serum proteins.

### SUMMARY OF THE INVENTION

The present invention relates to a method for determining the influence of human plasma or serum protein binding on antiretroviral activity at physiologically achieved conditions, by making use of specific virus strains in a cell-based antiviral assay.

The present invention provides as well a method for pharmacolinetically characterizing HIV inhibitors in the presence of plasma or serum proteins, that is by determining therapeutic amounts and subsequent dosage regimens, for various purposes including, lead optimisation, drug selection, preclinical evaluation, and clinical optimisation.

The present invention further concerns a method for selecting compounds that competitively bind with plasma or serum proteins as well as a method for selecting compounds that enhance binding of antiretrovirals with plasma or serum proteins, said selections being useful for co-administering agents to compete for or enhance plasma or serum protein binding, aiming to an improved management of therapeutic concentrations of HIV inhibitors.

### BRIEF DESCRIPTION OF THE DRAWINGS

FIG. 1 is a graph showing the influence of AAG on the anti-HIV activity of saquinavir

FIG. 2 is a graph showing the influence of AAG on the anti-HIV activity of indinavir

### DETAILED DESCRIPTION

The present invention relates to a method for determining the influence of human plasma or serum protein binding on antiretroviral activity. More specifically, the present invention provides a method for establishing the phenotypic variability of antiretroviral therapy in the presence of human plasma or serum proteins at physiologically achieved conditions, that is by maklng use of specific virus strains.

Thus, the present invention provides a method for determining the influence of human plasma or serum protein binding on antiretroviral therapy, comprising:
i) determining the inhibitory activity of at least one HIV inhibitor in a cellular assay in the presence of human plasma or serum proteins against at least one HIV virus strain;
ii) determining the inhibitory activity of the at least one HIV inhibitor in a cellular assay in the absence of the human plasma or serum proteins against the at least one HIV virus strain;
iii) calculating the ratio of inhibitory activities determined in i) and ii); and iv) determining the influence of human plasma or serum protein binding on said at least one HIV inhibitor based on the ratio obtained in iii); and
wherein said at least one HIV virus strain has been selected to be inhibited by said at least one HIV inhibitor with an inhibitory activity which falls within the range of plasma concentrations of said at least one HIV inhibitor when used at therapeutic dosages.

In a similar embodiment, the present invention provides a method for determining the influence of human plasma or serum protein binding on antiretroviral therapy, comprising:
i) determining the inhibitory activity of at least one protease inhibitor in a cellular assay in the presence of human plasma or serum proteins against at least one HIV virus strain;
ii) determining the inhibitory activity of the at least one protease inhibitor in a cellular assay in the absence of the human plasma or serum proteins against the at least one HIV virus strain;
iii) calculating the ratio of inhibitory activities determined in i) and id); and
iv) determining the influence of human plasma or serum protein binding on said at least one protease inhibitor based on the ratio obtained in iii); and
wherein said at least one HIV virus strain has been selected to be inhibited by said at least one protease inhibitor with an inhibitory activity which falls within the range of plasma concentrations of said at least one protease inhibitor when used at therapeutic dosages.

Any cell based assay capable of measuring changes in the ability of a pathogen or malignant cell to grow in the presence of a therapeutic agent(s) can be used in the present invention. Such assays of phenotyping include all methods known to persons of skill in the art. As an illustrative example, methods for phenotyping viruses suitable for use in the present invention include, but are not limited to, plaque reduction assays, PBMC p24 growth inhibition assays (see, e. g., Japour et al., Antimcrob. Agents Chemother. 37: 1095-1101 (1993); Kusumi et al., J. Virol. 66: 875-885 (1992), both of which are expressly incorporated herein by reference), recombinant virus assays (see, e. g., Kellam & Larder, Antimicrob. Agents Chemother. 38: 23-30 (1994); Hertogs, et al. Antimicrobial Agents and Chemotherapy (1998), 42(2), 269-276; and Pauwels et al., 2nd International Workshop on HIV Drug Resistance and Treatment Strategies, Laake Maggiore, Italy. Abstr. 51 (1998), all of which are expressly incorporated herein by reference); the use of GFP as a marker to assess the susceptibility of anti-viral inhibitors (Marschall et al., Institute of Clin. and Mol. Virol., University of Erlanger Nuremberg, Schlobgarten, Germany); and cell culture assays (Hayden et al., N. Eng. J. Med. 321: 1696-702 (1989), herein incorporated by reference).

As yet other illustrative examples, cellular assays for phenotyping malignant cells suitable for use in the present invention include, but are not limited to, flow cytometric assays (see, e. g., Pallis et al., Br. J. Haematol., 104 (2): 307-12 (1999); Huet et al., Cytometry 34 (6): 248-56 (1998), both of which are expressly incorporated herein by reference), fluorescence microscopy (see, e. g., Nelson et al., Cancer Chemother. Pharmacol. 42 (4): 292-9 (1998), expressly incorporated herein by reference), calcein accumulation method (see, e. g., Homolya et al., Br. J., Cancer. 73 (7): 849-55 (1996), expressly herein incorporated by reference), and ATP luminescence assay (see, e. g., Andreotti et al., Cancer Res. 55 (22): 5276-82 (1995), expressly incorporated herein by reference).

The measurement of the influence of plasma or serum protein binding on the efficacy, of various antiretrovirals may be used in concert with direct cell based phenotyping assays, for example, Antivirogram™ (Virco, Inc.; WO 97/27480, U.S. Pat. No. 6,221,578 incorporated herein by reference). Combined with HIV virus strains of various degrees of resistance or sensitivity, the present method allows the measurement of antiviral inhibitory activities in the presence and absence of plasma or serum proteins and determination of the efficacy of said antiretrovirals at physiological conditions.

Accordingly, the phenotypic drug sensitivity, or phenotypic drug resistance of the HIV virus strains to one or more HIV inhibitor(s) in the presence of plasma or serum proteins at physiological conditions is expressed as antiviral inhibitory activity, or effective concentrations. This is then compared to the inhibitory activity for the same assay components but in the absence of plasma or serum proteins. The phenotypic drug sensitivity or resistance of the sampled virus strain to each therapy in the presence of plasma or serum proteins is then expressed in terms of a fold-change in inhibitory activities, e.g. IC₅₀ values, IC₉₀ values, EC₅₀ values, EC90 values, etc., compared to the inhibitory activities obtained with the absence of said plasma or serum proteins.

“Susceptibility” or “sensitivity” to a therapy refers to the capacity of the disease, malignant cell, and/or pathogen to be affected by the therapy. “Resistance” refers to the degree to which the disease, malignant cell, and/or pathogen is unaffected by the therapy. The sensitivity, susceptibility or resistance of a disease towards a therapy may be expressed by means of an inhibitory activity or effective concentration value. The inhibitory activity is the concentration at which a given therapy results in a reduction of the pathogen's growth compared to the growth of the pathogen in the absence of a therapy. The effective concentration is the concentration of an inhibitor that produces the maximal possible effect As such, the EC₅₀ or EC₉₀ value is the effective drug concentration at which 50% or 90% respectively of the viral population is inhibited from replicating. The IC₅₀ or lC₉₀ value is the drug concentration at which 50% or 90% respectively of the enzyme activity is inhubited. Accordingly, other fractions are possible and range up to 100% such as, 90%, 75%, 50%, 30%, etc. Here in this invention, both terms will be referred as inhibitory activity.

Efficacy, also known as intrinsic activity, is used to describe the maximum effect of a drug.

In order to be able to test the influence of plasma or serum protein binding on the anti-HIV potency of compounds at physiologically achieved concentrations, thus at those concentrations of drug actually present in the patients, one needs to employ HIV viral strains of various degrees of resistance or sensitivity. Thus, viral strains for which the inhibitory activity of said anti-HIV compounds, when determined in the absence of human plasma or serum proteins, falls within the range of plasma drug concentrations of said compounds in patients. Accordingly, inhibitory activity values corresponding to clinically relevant concentrations, sub-therapeutic concentrations and in vitro testing concentrations are determined for the different viral strains, and those virus strains exhibiting clinically relevant inhibitory activity concentrations will be selected and employed in the methods of this invention. Preferably a maximum or worst-case measurable resistance should be obtained where a given inhibitor still has an effect. This concentration is dependent on the intrinsic activity of the drug and on the level of resistance of the virus strain.

Resistance of a disease to a therapy may be caused by alterations in phenotype or genotype. The resistant ‘behaviour’ of the virus is a combined result of the effects of many different mutations and the complex interactions between them, including genetic changes that have not even been identified yet. Genotypic alterations include mutations, single nucleotide polymorphisms, microsatellite variations, epigenetic variations such as methylation.

The influence of human plasma or serum protein binding on antiretroviral activity is defined as the change in inhibitory activities ratio, e.g. IC₅₀, IC₉₀, EC₅₀, EC₉₀ values ratios, the inhibitory activities being determined in the presence and in the absence of plasma or serum proteins. The influence of human plasma or serum protein binding on a certain drug is a measure of the variation of the potency exhibited by the drug.

Potency is a measure of the relative sensitivity on the concentration (or dose) axis in producing a particular effect.

Physiological conditions means the same range of therapeutic plasma concentrations as found in the human body. Physiological conditions also include those blood components at those concentrations that are found in the human body. For instance, physiological conditions comprises the addition of human serum at 50% concentration, alpha₁-acid glycoprotein in a range from 0.5 to 2 mg/ml, or human serum albumin at around 45 mg/ml.

Physiological plasma drug concentrations may be obtained from bibliographical data The different virus strains and level of resistance or sensitivity that they exhibit, in terms of increase of inhibitory activities, EC₅₀ values, or EC₉₀ values may also be found in the literature, or by running antiviral experiments. As an example, protease inhibitors have clinically relevant concentrations in a range of around 500 nM and more, sub-therapeutic concentrations in the range of around 50 nM to around 500 nM, and in vitro testing concentrations in the range of less than around or 50 nM. Viral strains, for which HIV inhibitors exhibit EC₅₀ values of interest for the methods of the present invention are for example R13020, T13127, T13025, LAI, for which saquinavir exhibits EC₅₀ values of around 7 μM, 710 nM, 166 nM, and 15-4.7 nM, respectively. For viral strains R13020, R13025, R13031, and LAI, ritonavir exhibits EC₅₀ values of around 27 μM, 2 μM, 407 nM, and 36 nM, respectively. For viral strains R13025, R13028, and LAI, indinavir exhibits EC₅₀ values of around 2 μM, 261 nM, and 36 nM, respectively. For viral strains R13127, R13036, R13028, and LAI, nelfinavir exhibits EC₅₀ values of around 8 μM, 1 μM, 254 nM, and 32 nM, respectively. For viral strains R13127, R13025, and LAI, amprenavir exhibits EC₅₀ values of around 2 μM, 412 nM, and 42 nM, respectively. For viral strains R13273, R13485, and LAI, lopinavir exhibits EC₅₀ values of around 2 μM, 367 nM, and 9 nM, respectively.

The term binding refers to an interaction or association between a minimum of two entities, or molecular structures, such as a ligand and an antiligand. The interaction may occur when the two molecular structures are in direct or indirect physical contact or when the two structures are physically separated but electromagnetically coupled there between, e.g. by hydrogen bonds or Van der Waals interactions. Examples of binding events of interest in a medical context include, but are not limited to, ligand/receptor, antigen/antibody, enzyme/substrate, enzyme/inhibitor, protein/protein, DNA/DNA, DNA/RNA, RNA/RNA, nucleic acid mismatches, complementary nucleic acids, nucleic acid/proteins, plasma or serum proteins/drugs, nucleic acids/drugs.

HIV inhibitors comprise nucleoside reverse transcriptase inhibitors (NRTIs), nucleotide reverse transcriptase inhibitors (NtRTIs), non-nucleoside reverse transcriptase inhibitors (NNRTIs), protease inhibitors (PIs), entry inhibitors, fusion inhibitors, gp41 inhibitors, gp120 inhibitors, integrase inhibitors, co-receptors inhibitors (e.g. CCRS, CXCR4, . . . ), budding/maturation inhibitors, etc.

HIV nucleoside reverse transcriptase inhibitors include those compounds whose mechanism of action comprises an inhibition of the viral reverse transcriptase enzyme. As example, and with no limitation to existing and future new compounds, HIV nucleoside reverse transcriptase inhibitors include zidovudine (AZT), lamivudine (3TC), stavudine (d4T), zalcitabine (ddC), didanosine (ddI), abacavir (ABC)

HIV non-nucleoside reverse transcriptase inhibitors include those compounds whose mechanism of action comprises an inhibition of the viral reverse transcriptase enzyme. As example, and with no limitation to existing and future new compounds, HIV non-nucleoside reverse transcriptase inhibitors include nevirapine, delavirdine, efavirenz, TMC120, TMC125, capravirine, calanolide, UC781, SJ-1366, benzophenones, PETT compounds, TSAO compounds.

HIV nucleotide reverse transcriptase inhibitors include those compounds whose mechanism of action comprises an inhibition of the viral reverse transcriptase enzyme. As example, and with no limitation to existing and future new compounds, HIV non-nucleotide reverse transcriptase inhibitors include adefovir (PMEA), tenofovir (PMPA), and other phosphonates.

HIV protease inhibitors include those compounds whose mechanism of action comprises an inhibition of the viral protease enzyme. As example, and with no limitation to existing and future new compounds, HIV protease inhibitors include ritonavir (RTV), indinavir (IDV), nelfinavir (NFV), amprenavir (APV), telinavir (SC-52151), tipranavir (TPV), saquinavir (SQV), lopinavir (LPV), atazanavir, palinavir, mozenavir, BMS 186316, DPC 681, DPC 684, AG1776, GS3333, KNI-413, KNI-272, L754394, L756425, LG-71350, PD161374, PD173606, PD177298, PD178390, PD178392, PNU 140135, maslinic acid, U-140690, R0033-4649, TMC114, TMC126, their prodrugs, metabolites, N-oxides and salts.

HIV entry inhibitors or gp120 inhibitors include those compounds whose mechanism of action comprises an inhibition of the viral entry or gp120 glycoprotein. As example, and with no limitation to existing and future new compounds, HIV entry or gp120 inhibitors include BMS806, dextran sulfate, suramin, chicoric acid.

HIV fusion inhibitors or gp41 inhibitors include those compounds whose mechanism of action comprises an inhibition of the viral fusion or gp41 glycoprotein. As example, and with no limitation to existing and future new compounds, HIV fusion or gp41 inhibitors include T20, T1249.

HIV integrase inhibitors include those compounds whose mechanism of action comprises an inhibition of the viral integrase enzyme. As example, and with no limitation to existing and future new compounds, HIV integrase inhibitors include L-870810, L-870812, pyranodipyrimydines, S-1360.

Co-receptors inhibitors include those compounds whose mechanism of action comprises an inhibition of the interaction of HIV with cellular receptors present on the cell membrane (e.g. CCR5, CXCR4). As example, and with no limitation to existing and future new compounds, co-receptors inhibitors include TAK 779, AMD3100, AMD8664, AMD070, SHC-C, SHC-D, AK602, TAK-220, UK-427,857, T22.

HIV budding/maturation inhibitors include those compounds whose mechanism of action comprises an inhibition of the viral budding/maturation. As example, and with no limitation to existing and future new compounds, HIV budding/maturation inhibitors include PA-457.

By HIV herein is generally meant HIV-1. However, the invention is also applicable to HIV-2.

Plasma or serum proteins include all proteins found endogenously in plasma or serum. Examples of plasma or serum proteins include without limitation Albumin (HSA), Alpha-1-acid Glycoprotein (AAG), Alpha-1-Antichymotrypsin, Alpha-1 Antitrypsin AT, alpha-fetoprotein, Alpha-1-microglobulin A1M, Alpha-2-Macroglobulin A2M, Angiostatin, Beta-2-Glycoprotein 1, Beta-2-microglobulin, Beta-2-Microglobulin B2M, Beta-N-Acetylglucosaminidase B-NAG, recombinant Centromere Protein B, Collagens (type 1-VI), Complement Clq, Complement C3, Complement C4, Ceruplasmin, Chorionic Gonadotrophin HCG, Chorionic Gonadotrophin Beta CORE BchCG, C-Reactive Protein CRP, CK-MB (Creatine Kinase-MB), CK-MM & CK-BB, Cystatin C, D-Dimer, dsDNA, Ferritins, Glycogen Phosphorylase ISO BB, Haptoglobulin, IgA, IgE, IgG, IgG, IgM, Kappa light chain, lambda light chain, recombinant LKM Antigen, La/SS-B, Lysozyme, Myelin Basic Protein, Myoglobin, Neuron-Specific Enolase, Placental Lactogen, Prealbumin, Pregnancy assoc Plasma Protein A, Pregnancy specific beta 1 glycoprotein (SP1), Prostate Specific Antigen PSA, PSA-A1-Act complex, Prostatic Acid Phosphatase PAP, Proteinase 3 (PR3/Anca), Prothrombin, Retinol Binding Globulin RBP, recombinant human RO/SS-A 52 kda, recombinant human RO SS-A 60 kda, Sex Hormone Binding Globulin SHBG, S100 (BB/AB), S100 BB homodimer, Thyroglobulin Tg, Thyroid Microsomal Antigen, recombinant thyroid peroxidase TPO, Thyroid Peroxidase TPO, Thyroxine Binding Globulin TBG, Transferrin, Transferrin receptor, Troponin I complex, Troponin C, Troponin I, Troponin T, Urine Protein 1.

Plasma or serum proteins may also encompass proteins of external origin, which are not necessarily forming part of the common physiological population, but may be found in the body, i.e. proteins from diet origin or from drug compositions. Preferably, plasma or serum proteins are human α₁-acid glycoprotein (AAG), human serum albumin (HSA), human serum (HS), and lipoproteins, which are proteins mostly involved in the binding of HIV antivirals in plasma or serum Human AAG is an acute-phase protein whose expression increases during acute inflammatory episodes, infections, injuries, neoplastic disease, and AIDS (Kremer et al, Pharmacol Rev. 1988, 40: 1-47; Oie et al, 1993, J Acquir Immune Defic Syndr Hum Retrovirol. 5:531-533; Mackiewicz et al, 1995, Glycoconj. J. 12:241-247; van Dijk et al, 1995, Glycoconj J. 12:227-233). The level of AAG in human serum fluctuates between 0.15 and 1.5 mg/mL, and the average value may vary by as much as 4-fold between healthy volunteers and AIDS patients (Kremer et al, Pharnacol Rev. 1988, 40: 1-47; Oie et al, 1993, J Acquir Immune Defic Syndr Hum Retrovirol. 5:531-533). Additionally, AAG concentrations have been suggested to vary by race or ethnicity (Johnson et al, 1997, J. Pharm. Sci. 86: 1328-1333). It has been reported that AAG exists as a mixture of two or three genetic variants (the A variant and the F1 and/or S variants) in the plasma or serum of most individuals (Herve et al, 1998, Mol. Pharmacol. 54:129-138), which present different drug binding specificities.

Human serum albumin (HSA) is quite an abundant protein in the blood stream—it is present at a concentration of about 40 mg/ml. The primary function of HSA is to act as a transporter of fatty acid molecules. HSA has multifunctional binding properties which range from various metals, calcium and copper, to fatty acids, hormones and therapeutic drugs.

Particular plasma or serum proteins may have several variants. The term protein variant refers to a polypeptide comprising one or more substitutions of the specified amino acid residues underlying the protein. The total number of such substitutions is typically not more than 10, e.g. one, two, three, four, five or six of said substitutions. In addition, the protein variant may optionally include other modifications of the parent enzyme, typically not more than 10, e.g. not more than 5 such modifications. The variant generally has a homology with the parent enzyme of at least 80%, e.g. at least 85%, typically at least 90% or at least 95%. Variants may not only differ in primary structure (amino acid sequence), but also in secondary or tertiary structure and the amount and structure of covalently attached carbohydrates. A protein may be present in plasma or serum in different variants, at similar or different concentrations. Variants of a protein may exhibit different binding properties. For instance human α-1 acid glycoprotein is present in two different variants, A and F1/S, which have different binding properties to various ligands and drugs. Human serum albumin may be present as different mutants such as K195M, K199M, F211V, W214L, R218M, R222M, H242V, and R257M.

The methods of the present invention may additionally comprise as part of the test composition, any compound, including, but not limited to, dipeptides, tripeptides, polypeptides, proteins, small and large organic molecules, buffers, or test aid components, and derivatives thereof.

In a particular embodiment, the method preferably includes a competitive binding agent. A competitive binding agent refers to those molecules that competitively bind to plasma or serum proteins in the presence of HIV inhibitors. Said competitive binding agent could be one or two more drugs, preferably other drugs than antivirals which bind to plasma or serum proteins, also preferably one or two more drugs effective to treat AIDS and related syndromes, more preferably one or two more antivirals, such as NNRTI, NRTI, PI, fusion inhibitors, entry inhibitors, integrase inhibitors, as well preferably, the compound ritonavir, so concomitant administration of antiretrovirals, optionally with other types of drugs, and its influence on plasma or serum protein binding properties may be studied. Therefore, the present invention also provides a method for identifying compounds that bind competitively to plasma or serum proteins in the presence of HIV inhibitors. Said compounds may be used as co-administered agents to increase the free plasma or serum concentration of protease inhibitors.

In an alternative embodiment, the method includes a binding enhancing agent. A binding enhancing agent refers to those molecules that enhance the binding of antiretrovirals to plasma or serum proteins. Thus, the present invention provides a method for identifying compounds that enhance the binding of HIV inhibitors to plasma or serum proteins. Said compounds may be used as co-administered agents to improve management of therapeutic concentrations of HIV inhibitors.

The inhibitory activities, fold-changes and ratios thereof, exhibited by the different HIV inhibitors in the presence of various viral strains, and in the presence and absence of plasma or serum proteins, are particularly useful values to establish the pharmacokinetic characteristics of the antivirals such as the distribution volume, half-life, bioavailability, and to further determine the therapeutic amount, dosage amounts and dosage intervals, necessary to accomplish an effective therapeutic treatment. Same information may be used for patient management, therapeutic drug monitoring, thus, to adjust and tailor the dosage regimen for individual patients and conditions. Thus, the present invention further provides a method for pharmacokinetically characterizing HIV inhibitors, comprising:
i) determining the inhibitory activity of at least one HIV inhibitor in a cellular assay in the presence of human plasma or serum proteins against at least one HIV virus strain;
ii) determining the inhibitory activity of the at least one HIV inhibitor in a cellular assay in the absence of the human plasma or serum proteins against the at least one HIV virus strain;
iii) calculating the ratio of inhibitory activities determined in i) and ii);
iv) determining the inhibitory activity of the at least one HI inhibitor against at least one HIV virus strain of a patient;
v) multiplying the ratio obtained in iii) by the inhibitory activity determined in iv); and
vi) using the inhibitory activity as determined in v) to calculate physiological therapeutic dosages; and
wherein said at least one HIV virus strain in i) and ii) has been selected to be inhibited by said at least one HIV inhibitor with an inhibitory activity which falls within the range of plasma concentrations of said at least one HIV inhibitor when used at therapeutic dosages.

In a similar embodiment, the present invention provides a method for pharmacokinetically characterizing protease inhibitors, comprising:
i) determining the inhibitory activity of at least one protease inhibitor in a cellular assay in the presence of human plasma or serum proteins against at least one HIV virus strain;
ii) determining the inhibitory activity of the at least one protease inhibitor in a cellular assay in the absence of the human plasma or serum proteins against the at least one HIV virus strain;
iii) calculating the ratio of inhibitory activities determined in i) and ii);
iv) determining the inhibitory activity of the at least one protease inhibitor against at least one HIV virus strain of a patient;
v) multiplying the ratio obtained in iii) by the inhibitory activity determined in iv); and
vi) using the inhibitory activity as determined in v) to calculate physiological therapeutic dosages; and
wherein said at least one HIV virus strain in i) and ii) has been selected to be inhibited by said at least one protease inhibitor with an inhibitory activity which falls within the range of plasma concentrations of said at least one protease inhibitor when used at therapeutic dosages.

The present invention further provides a method of constructing a pharmacokinetic profile database of HIV inhibitors, with the influence of plasma or serum protein binding, comprising:
i) determining the inhibitory activity of at least one HIV inhibitor in a cellular assay in the presence of human plasma or serum proteins against at least one HIV virus strain;
ii) determining the inhibitory activity of the at least one HIV inhibitor in a cellular assay in the absence of the human plasma or serum proteins against the at least one HIV virus strain;
iii) calculating the ratio of inhibitory activities determined in i) and ii);
iv) determining the influence of human plasma or serum protein binding on said at least one HIV inhibitor based on the ratio obtained in iii);
v) determining the inhibitory activity of the at least one HIV inhibitor against at least one HIV virus strain of a patient;
vi) multiplying the ratio obtained in iii) by the inhibitory activity determined in v),
vii) using the inhibitory activity as determined in v) to calculate physiological therapeutic dosages; and
viii) correlating in a data table the influence of human plasma or serum protein binding of HIV inhibitors as determined in iv) with the physiological therapeutic dosages as determined in vii); and
wherein said at least one HIV virus strain in i) and ii) has been selected to be inhibited by said at least one HIV inhibitor with an inhibitory activity which falls within the range of plasma concentrations of said at least one HIV inhibitor when used at therapeutic dosages.

Said method for constructing such database also encompasses reports that are generated comprising a listing, analysis, or other information regarding the influence of plasma or serum protein binding, pharmacokinetic parameters, and their correlation to drug dosage regimens.

The method of this invention has further applications in the preclinical evaluation and selection of new antivirals for future clinical development. As such, the present invention additionally comprises a method for measuring the influence of plasma or serum protein binding on new compounds, comprising:
i) determining the inhibitory activity of at least one HIV inhibitor in a cellular assay in the presence of human plasma or serum proteins against at least one HIV virus strain;
ii) determining the inhibitory activity of the at least one HIV inhibitor in a cellular assay in the absence of the human plasma or serum proteins against the at least one HIV virus strain;
iii) calculating the ratio of inhibitory activities determined in i) and ii); and
iv) determining the influence of human plasma or serum protein binding on said at least one HIV inhibitor based on the ratio obtained in iii); and
wherein said at least one HIV virus strain has been selected to be inhibited by said at least one HIV inhibitor with an inhibitory activity which falls within the range of plasma concentrations of said at least one HIV inhibitor when used at therapeutic dosages.

The methods provided in the present invention may optionally be used as or comprise part of a high-throughput screening assay where numerous test compositions are evaluated for the effect of plasma or serum protein binding to HIV inhibitors and for the pharmacokinetic derived properties of said HIV inhibitors.

The order of the steps of the methods of the invention may be varied. One of skill in the art would be able to determine which variations in the order of the steps are applicable.

Other embodiments of the invention will be apparent to those skilled in the art from consideration of the specification and practice of the invention disclosed herein. It is intended that the specification and examples be considered as exemplary only, with a true scope and spirit of the invention being indicated by the claims.

### EXAMPLE 1

### Influence of Human Plasma or Serum Proteins on Activity of Current PIs Against Wild-Type HIV-1

The influence of AAG, Human Serum Albumin (HSA) or Human Serum (HS) on the activity of antiretrovirals was measured in a cell-based antiviral assay. A phenotypic assay was performed to measure the ability of resistant virus strains to grow in the presence of each drug of interest, and in the presence of various combinations of plasma or serum proteins at different concentrations:
50% heat-inactivated human serum (HS) with or without 10% fetal calf serum (FCS)
1 mg/mL AAG and 10% FCS
45 mg/mL human serum albumin (HSA) and 10% FCS

Anti-HIV activity was determined in HIV-or mock-infected MT4 cells by the MTT method as described by Pauwels et al (1988), J.Virol.Meth.20:399-321, and Hertogs et al (1998), Antimicrob.Ag.Chemother.42:269-27. Briefly, recombinant or non-recombinant virus, with a specific resistance profile, were grown in cell culture to obtain viral stocks of known concentration. Susceptibility testing of the viral stocks in the presence of various antiviral agents and in the presence of plasma or serum proteins, and a detection system based on MTT determined to which extent agents inhibited replication of the virus strains.

Virus strains used for these studies had different origins:
HIV strains obtained by in vitro selection in the presence of various antivirals
Recombinant clinical isolates constructed according to the Antivirogram™ method as described by Hertogs et al (1998).

With the exception of indinavir, which remained unaffected, all tested PIs showed a decrease in potency against wild-type HIV-1 in the presence of AAG and HS, but not of HSA. The decrease ranged from 5-to 75-fold and was proportional to the AAG concentration. Virus strains with various levels of resistance against each of the PIs were used in similar experiments with AAG. The results obtained showed that the decrease in potency observed in the presence of AAG for the tested PIs was inversely proportional to the EC₅₀ value in the absence of the protein. At micromolar concentrations (1-5 μM), saquinavir, ritonavir, nelfinavir, amprenavir and compound 1 showed only a less than 5-fold decrease in potency in the presence of 1 mg/mL AAG. So it was concluded that the influence of AAG decreased with increasing concentrations of drugs.

TABLE 1
Influence of Human Plasma Proteins on Activity of Current PIs
against Wild Type HIV-1
Results in table 1 are expressed as the ratio between the EC₅₀
value determined in the presence of the indicated human plasma
or serum protein and the EC₅₀ value determined in the
presence of 10% FCS, against wild type HIV-1 (LAI). Results are
median of at least two determinations.

| | FCS | | | |
|---|---|---|---|---|
| Compound | (10%) | AAG 1 mg/mL | HSA 45 mg/mL | HS 50% |
| INDINAVIR | 1 | 1 | 2 | 3 |
| SAQUINAVIR | 1 | 5 | 3 | 5 |
| NELFINAVIR | 1 | 31 | 7 | 25 |
| RITONAVIR | 1 | 18 | 5 | 24 |
| LOPINAVIR | 1 | 26 | 5 | 5 |
| AMPRENAVIR | 1 | 25 | 2 | 12 |

TABLE 2
EC₅₀ ratios, EC₅₀values determined in the presence of human
plasma or serum protein and in the presence of 10% fetal calf
serum (FCS), against various resistant strains.
Results in table 2 are expressed as the ratio between the EC₅₀
value determined in the presence of the indicated human
plasma or serum protein and the EC₅₀ value determined in
the presence of 10% FCS, against various HIV-1 strains.
Results are median of at least two determinations.

| | | | | in vitro testing | |
|---|---|---|---|---|---|
| | clinically relevant | | | concentrations | |
| | concentrations | | sub-therapeutic | (wild type HIV) | |
| concentration | | 5 μM- | concentrations | | <5 |
| range | >5 μM | 500 nM | 500 nM-50 nM | 50 nM-5 nM | nM |
| Saquinavir | 2 | 3 | 4 | 5 | 19 |
| Ritonavir | 1 | 4 | 8 | 7 | — |
| Indinavir | — | 2 | 1 | 2 | — |
| Nelfinavir | — | 5 | 9 | 11 | — |
| amprenavir | — | 5 | 14 | 38 | — |
| Lopinavir | — | 4 | 20 | 64 | — |
| compound 1 | 4 | 7 | 12 | 24 | 85 |

Compound 1 is a PI with the following formula:

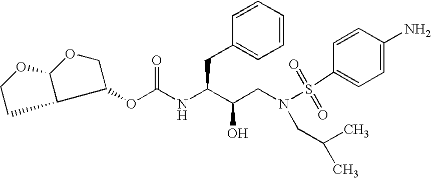

### EXAMPLE 2

### Influence of AAG on the Anti-HIV Activity of Saquinavir (SQV), a Highly Protein-Bound PI

Saquinavir was tested against different HIV-1 strains with various susceptibility (EC₅₀ values) to the inhibitor in the indicated concentration range. One curve represents the data obtained in the presence of 10% FCS. One curve represents the data calculated for the different strains based on the decrease observed for SQV activity against wild type HIV-1 in the presence of AAG (1 mg/mL). Another curve represents the data obtained for the different strains in the presence of AAG (1 mg/mL).

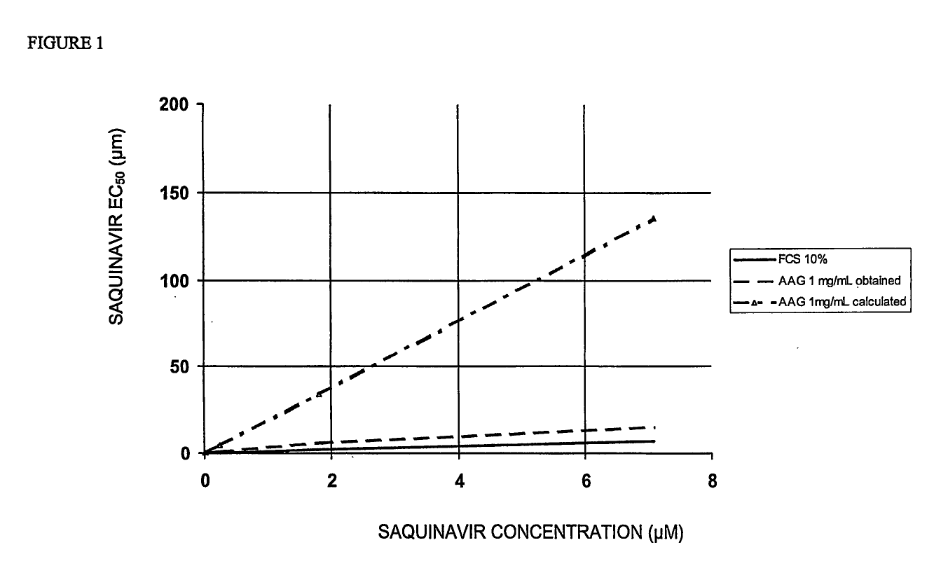

See FIG. 1.

The influence of AAG on the anti-HIV activity of SQV decreases with increasing inhibitor concentrations.

### EXAMPLE 3

### Influence of AAG on the Anti-HIV Activity of Indinavir, a Lowly Protein-Bound PI

Indinavir was tested against different HIV-1 strains with various susceptibility (EC₅₀ values) to the inhibitor in the indicated concentration range. One curve represents the data obtained in the presence of 10% FCS. One curve represents the data calculated for the different strains based on the decrease observed for IDV activity against wild type HIV-1 in the presence of AAG (1 mg/mL). Another curve represents the data obtained for the different strains in the presence of AAG (1 mg/mL).

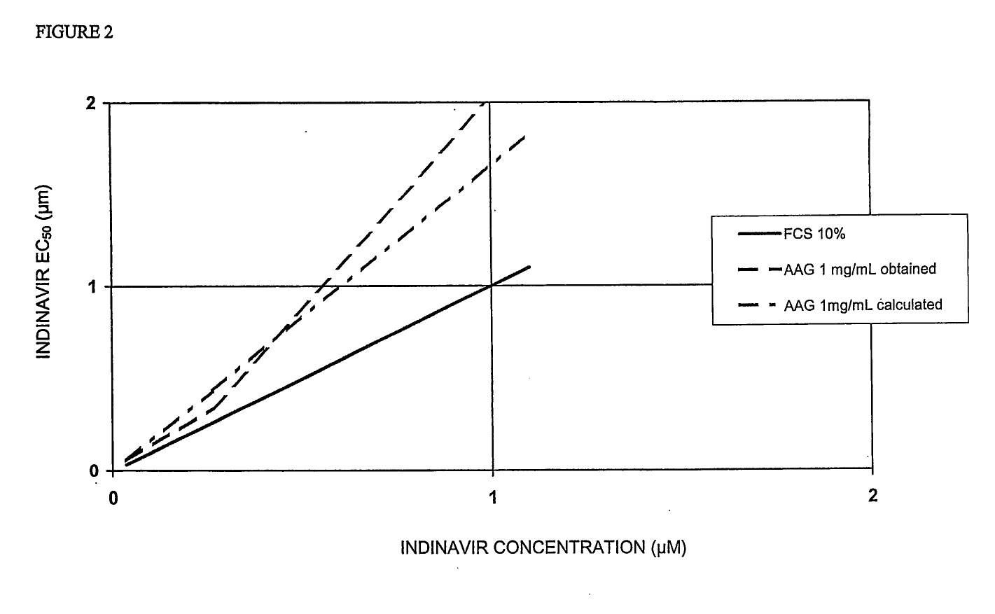

See FIG. 2.

At clinically relevant inhibitor concentrations, the influence of AAG on anti-HIV activity of PIs is minimized, thereby showing the saturation of the interaction of the drugs with the protein.

1. A method for determining the influence of human plasma or serum protein binding on antiretroviral therapy, comprising:
i) determining the inhibitory activity of at least one HIV inhibitor in a cellular assay in the presence of human plasma or serum proteins against at least one HIV virus strain;
ii) determining the inhibitory activity of the at least one HIV inhibitor in a cellular assay in the absence of the human plasma or serum proteins against the at least one HIV virus strain;
iii) calculating the ratio of inhibitory activities determined in i) and ii); and
ii) determining the influence of human plasma or serum protein binding on said at least one HIV inhibitor based on the ratio obtained in iii); and
wherein said at least one HIV virus strain has been selected to be inhibited by said at least one HIV inhibitor with an inhibitory activity which falls within the range of plasma concentrations of said at least one HIV inhibitor when used at therapeutic dosages.

2. A method for determining the influence of human plasma or serum protein binding on antiretroviral therapy, comprising:
i) determining the inhibitory activities of at least one protease inhibitor in a cellular assay in the presence of human plasma or serum proteins against at least one HIV virus strain;
ii) determining the inhibitory activities of the at least one protease inhibitor in a cellular assay in the absence of the human plasma or serum proteins against the at least one HIV virus strain;
iii) calculating the ratio of inhibitory activities determined in i) and ii); and
iii) determining the influence of human plasma or serum protein binding on said at least one protease inhibitor based on the ratio obtained in iii); and
wherein said at least one HIV virus strain has been selected to be inhibited by said at least one protease inhibitor with an inhibitory activity which falls within the range of plasma concentrations of said at least one protease inhibitor when used at therapeutic dosages.

3. A method according to claim 1, wherein the plasma or serum proteins are chosen from human serum, albumin, α₁-acid glycoprotein, lipoproteins, and variants thereof.

4. A method according to claim 1, wherein the method further comprises at least one competitive binding agent or at least one binding enhancing agent.

5. A method of identifying compounds that bind competitively to plasma or serum proteins in the presence of HIV inhibitors, said method based on determining the influence of human plasma or serum protein binding on antiretroviral therapy according to claim 1.

6. A method of identifying compounds that enhance binding of HIV inhibitors to plasma or serum proteins, said method based on determining the influence of human plasma or serum protein binding on antiretroviral therapy according to claim 1.

7. A method for pharmacologically characterizing HIV inhibitors comprising:
i) determining the inhibitory activity of at least one HIV inhibitor in a cellular assay in the presence of human plasma or serum proteins against at least one HIV virus strain;
ii) determining the inhibitory activity of the at least one HIV inhibitor in a cellular assay in the absence of the human plasma or serum proteins against the at least one HIV virus strain;
iii) calculating the ratio of inhibitory activities determined in i) and ii);
iv) determining the inhibitory activity of the at least one HIV inhibitor against at least one HIV virus strain of a patient;
v) multiplying the ratio obtained in iii) by the inhibitory activity determined in iv); and
vi) using the inhibitory activity as determined in v) to calculate physiological therapeutic dosages; and
wherein said at least one HIV virus strain in i) and ii) has been selected to be inhibited by said at least one HIV inhibitor with an inhibitory activity which falls within the range of plasma concentrations of said at least one HIV inhibitor when used at therapeutic dosages.

8. A method for pharmacokinetically characterizing protease inhibitors comprising:
i) determining the inhibitory activity of at least one protease inhibitor in a cellular assay in the presence of human plasma or serum proteins against at least one HIV virus strain;
ii) determining the inhibitory activity of the at least one protease inhibitor in a cellular assay in the absence of the human plasma or serum proteins against the at least one HIV virus strain;
iii) calculating the ratio of inhibitory activities determined in i) and ii);
iv) determining the inhibitory activity of the at least one protease inhibitor against at least one HIV virus strain of a patient;
v) multiplying the ratio obtained in iii) by the inhibitory activity determined in iv); and
vi) using the inhibitory activity as determined in v) to calculate physiological therapeutic dosages; and
wherein said at least one HIV virus strain in i) and ii) has been selected to be inhibited by said at least one protease inhibitor with an inhibitory activity which falls within the range of plasma concentrations of said at least one protease inhibitor when used at therapeutic dosages.

9. A method of constructing a pharmacokinetic profile database of HIV inhibitors, with the influence of plasma or serum protein binding, comprising:
i) determining the inhibitory activity of at least one HIV inhibitor in a cellular assay in the presence of human plasma or serum proteins against at least one HIV virus strain;
ii) determining the inhibitory activity of the at least one HIV inhibitor in a cellular assay in the absence of the human plasma or serum proteins against the at least one HIV virus strain;
iii) calculating the ratio of inhibitory activities determined in i) and ii);
iv) determining the influence of human plasma or serum protein binding on said at least one HIV inhibitor based on the ratio obtained in iii);
V) determining the inhibitory activity of the at least one HIV inhibitor against at least one HIV virus strain of a patient;
vi) multiplying the ratio obtained in iii) by the inhibitory activity determined in v);
vii) using the inhibitory activity as determined in v) to calculate physiological therapeutic dosages; and
viii) correlating in a data table the influence of human plasma or serum protein binding of HIV inhibitors as determined in iv) with the physiological therapeutic dosages as determined in vii); and
wherein said at least one HIV virus strain in i) and ii) has been selected to be inhibited by said at least one HIV inhibitor with an inhibitory activity which falls within the range of plasma concentrations of said at least one HIV inhibitor when used at therapeutic dosages.

10. A method for measuring the influence of plasma or serum protein binding on new compounds comprising:
i) determining the inhibitory activity of at least one HIV inhibitor in a cellular assay in the presence of human plasma or serum proteins against at least one HIV virus strain;
ii) determining the inhibitory activity of the at least one HIV inhibitor in a cellular assay in the absence of the human plasma or serum proteins against the at least one HIV virus strain;
iii) calculating the ratio of inhibitory activities determined in i) and ii); and
iv) determining the influence of human plasma or serum protein binding on said at least one HIV inhibitor based on the ratio obtained in iii); and
wherein said at least one HIV virus strain in has been selected to be inhibited by said at least one HIV inhibitor with an inhibitory activity which falls within the range of plasma concentrations of said at least one HIV inhibitor when used at therapeutic dosages.

11. A method according to claim 1 suitable for high throughput screening.

12. The method according to claim 2, wherein the plasma or serum proteins are chosen from human serum, albumin, α1-acid glycoprotein, lipoproteins, and variants thereof.

13. The method according to claim 2, wherein the method further comprises at least one competitive binding agent or at least one binding enhancing agent.

14. The method according to claim 3, wherein the method further comprises at least one competitive binding agent or at least one binding enhancing agent.

15. The method of identifying compounds that bind competitively to plasma or serum proteins in the presence of HIV inhibitors, said method based on determining the influence of human plasma or serum protein binding on antiretroviral therapy according to claim 2.

16. The method of identifying compounds that bind competitively to plasma or serum proteins in the presence of HIV inhibitors, said method based on determining the influence of human plasma or serum protein binding on antiretroviral therapy according to claim 3.

17. The method of identifying compounds that bind competitively to plasma or serum proteins in the presence of HIV inhibitors, said method based on determining the influence of human plasma or serum protein binding on antiretroviral therapy according to claim 4.

18. The method of identifying compounds that bind competitively to plasma or serum proteins in the presence of HIV inhibitors, said method based on determining the influence of human plasma or serum protein binding on antiretroviral therapy according to claim 12.

19. The method of identifying compounds that bind competitively to plasma or serum proteins in the presence of HIV inhibitors, said method based on determining the influence of human plasma or serum protein binding on antiretroviral therapy according to claim 13.

20. The method of identifying compounds that bind competitively to plasma or serum proteins in the presence of HIV inhibitors said method based on determining the influence of human plasma or serum protein binding on antiretroviral therapy according to claim 14.

21. The method according to claim 2 suitable for high throughput screening.

22. The method according to claim 3 suitable for high throughput screening.

23. The method according to claim 4 suitable for high throughput screening.

24. The method according to claim 5 suitable for high throughput screening.

25. The method according to claim 6 suitable for high throughput screening.

26. The method according to claim 7 suitable for high throughput screening.

27. The method according to claim 8 suitable for high throughput screening.

28. The method according to claim 9 suitable for high throughput screening.

29. The method according to claim 10 suitable for high throughput screening.

30. The method according to claim 11 suitable for high throughput screening.

31. The method according to claim 12 suitable for high throughput screening.

32. The method according to claim 13 suitable for high throughput screening.

33. The method according to claim 14 suitable for high throughput screening.

34. The method according to claim 15 suitable for high throughput screening.

35. The method according to claim 16 suitable for high throughput screening.

36. The method according to claim 17 suitable for high throughput screening.

37. The method according to claim 18 suitable for high throughput screening.

38. The method according to claim 19 suitable for high throughput screening.

39. The method according to claim 20 suitable for high throughput screening.
